(19) United States
(12) Patent Application Publication
(10) Pub. No.: US 2005/0283176 A1
(43) Pub. Date: Dec. 22, 2005

(54) Advanced disposable microdermabrasion system/method of treating the skin surface

(76) Inventor: Wahson Law, Livingston, NJ (US)

Correspondence Address: Washon Law, 44 Park Drive, Livingston, NJ, 07039, US

(21) Appl. No.: 10/878,137

(22) Filed: Jun. 22, 2004

(51) Int. Cl. A61B 17/14

(52) U.S. Cl. 606/180

(57) ABSTRACT

This invention provides a skin treatment device and disposable tissue collection/nutrition system, for removing of dead cells and outer layers of skin to provide a shining and fresh skin surface. Especially, the disposable scrap collection tip helps to minimize the probability of cross-infection among patients, and the nutrition system provides immediate nutrition supply to the fresh skins.

### CROSS-REFERENCE TO RELATED APPLICATIONS

U.S. patent Documents:
U.S. Pat. No. 2,712,823 July 1955 Kurtin,
U.S. Pat. No. 2,867,214 January 1959 Wilson,
U.S. Pat. No. 2,881,763 April 1959 Robbins,
U.S. Pat. No. 3,964,212 June 1983 Cortese, Jr.,
U.S. Pat. No. 4,957,747 September 1990 Stiefel,
U.S. Pat. No. 5,012,797 May 1991 Liang et Al.,
U.S. Pat. No. 5,037,432 August 1991 Smmers et al. 606/131
U.S. Pat. No. 5,100,412 March 1992 Rosso. 128/898
U.S. Pat. No. 5,800,446 September 1998 Banuchi. 606/131
U.S. Pat. No. 5,810,842 September 1998 Di Fiore et al. 606/131
U.S. Pat. No. 5,971,999 October 1999 Naldoni. 606/131
U.S. Pat. No. 6,042,552 March 2000 Cornier. 600/562
U.S. Pat. No. 6,241,793 June 2001 Waldron
   Other Publications:
News Release, American Society for Dermatologic Surgery, An Update on Micro-Dermabrasion, May 19, 1999 pp. 1-2.
Nidecker, Anna, “Microdermabrader Offers Alternative to Laser, Peels”, [Skin & Allergy News 29(3):48, 1998 International Medical News Group], pp. 1-2.

### BACKGROUND OF THE INVENTION

Dermabrasion, also referred to as microdermabrasion, is a process for removal of dead cells from the outermost layer of the skin, known as the epidermis, clean out blocked skin pores, and thus improve the healthiness of skins. In addition, it helps to erase traumatic scars such as acne scars and other damaged skin spots, such as aging spots or sun-burned spots. Without touching the lower two skin layers, the dermis and the subcutaneous layer, it can polish the surface of the skin and gives the patient a fresh and shining look. The process typically last for 20 minutes. Patients are suggested to take such treatment as periodic maintenance sessions, to improve the condition of skin generally.

Many methods, tools and treatment systems have been developed to remove outer layers of skin to provide a smooth and polished surface of skin. The objective is to remove undesirable dead skin cells without any potential side effects. However, these methods, tools and treatment systems are stereotyped and can only have very basic functions. In addition, these tools and treatments increase the possibility of cross-infection among patients.

### BRIEF SUMMARY OF THE INVENTION

This invention provides a skin treatment device and disposable tissue collection/nutrition system, for removing of dead cells and outer layers of skin to provide a shining and fresh skin surface. Especially, the disposable scrap collection tip helps to minimize the probability of cross-infection among patients, and the nutrition system provides immediate nutrition supply to the fresh skins. Especially, the disposable tip system minimizes the probability of cross-infection among patients; improve the usability and safety of the system.

The benefits of the advanced disposable microdermabrasion system are:
1. Greatly minimize the potential risk of cross-infection among patients;
2. Poor, dim skin is enhanced by a fresh surface of the superficial skin layers;
3. Fresh skins is provided with nutrition such as oxygen immediately;
4. Expression lines typically seen on forehead, eyes and mouth are softened;
5. Superficial damaged marks such as acne marks, scratch marks, burn spots can be smoothed;

### BRIEF DESCRIPTION OF THE SEVERAL VIEWS OF THE DRAWING

See FIG. 1.1, FIG. 1.2 and FIG. 2.

FIG. 1.1 The disposable tip system: Theoretical drawing of the tip system.

FIG. 1.2 The disposable tip can have various shapes: A professional design sample of a tip system.

FIG. 2 The tube system: Theoretical drawing of the tube system.

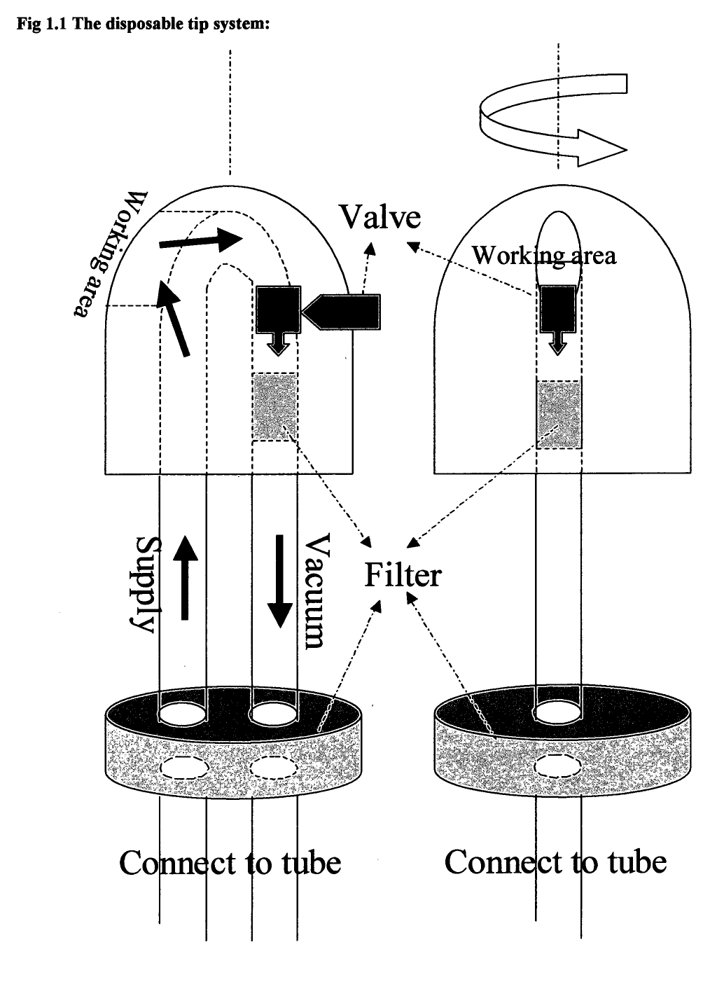

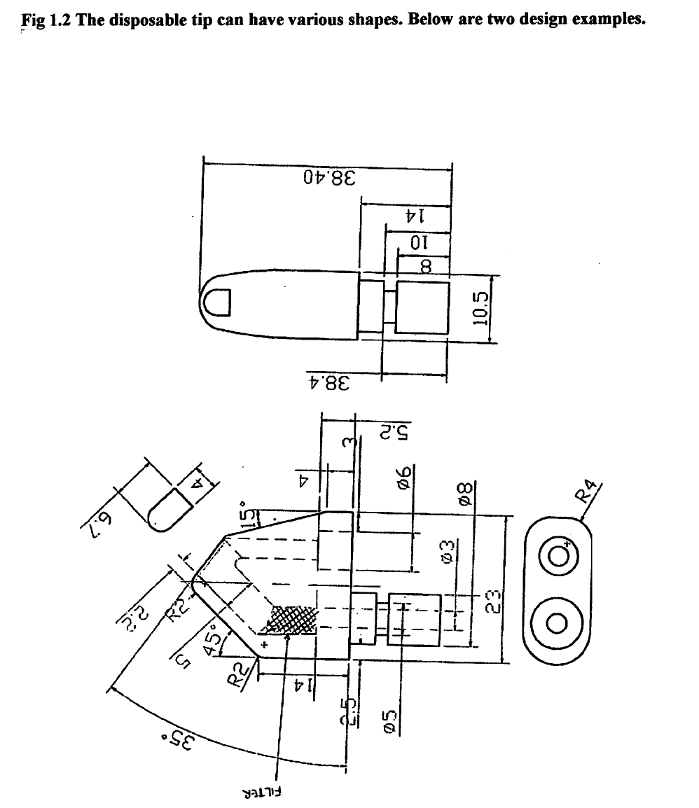

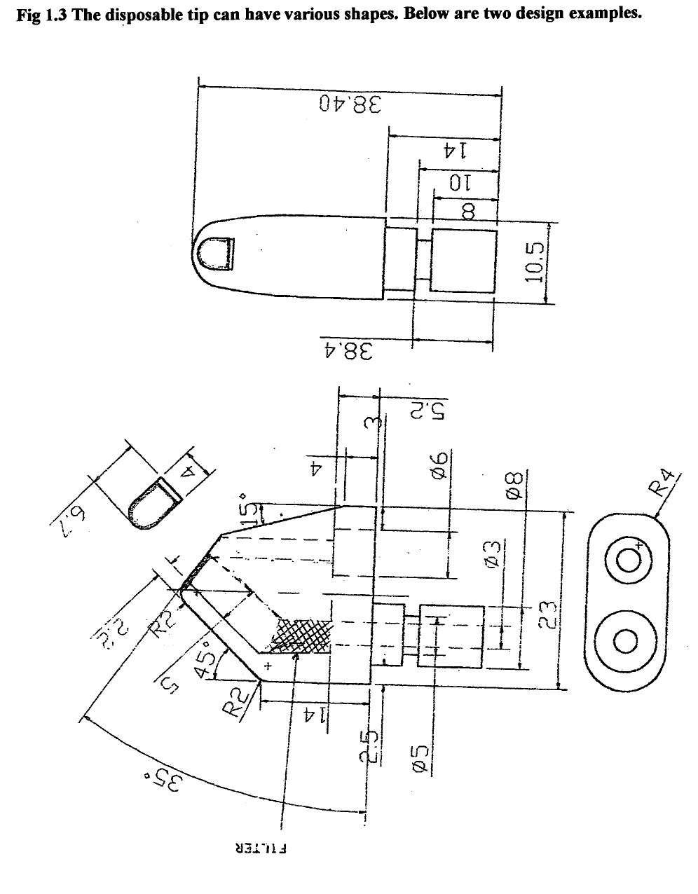

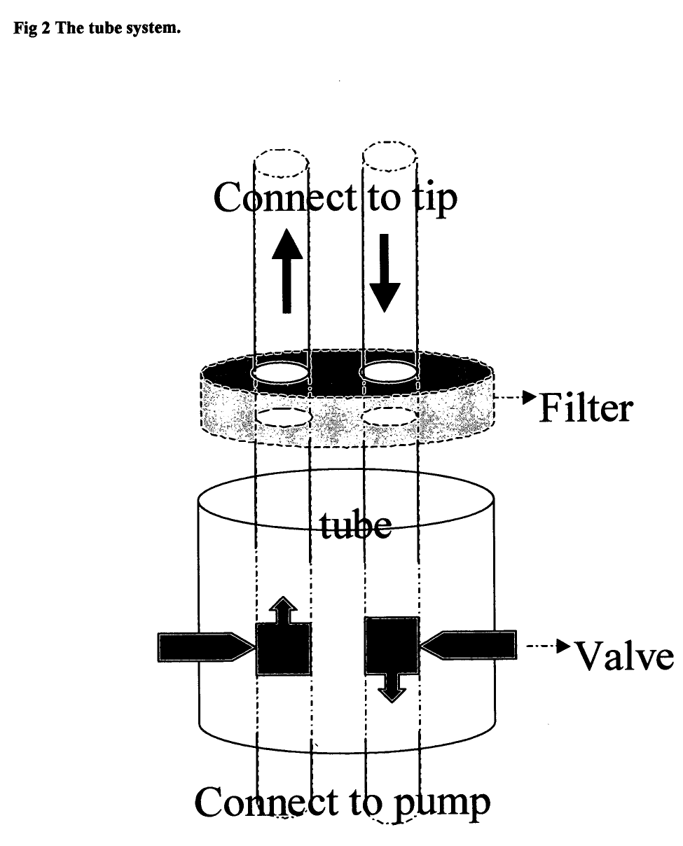

### DETAIL DESCRIPTION OF THE INVENTION

The microdermabrasion system comprises a vacuum/supply housing, a hollow tube and an abrasive tip attached to a skin-contacting end. The abrasive coated tip is moved over the skin surface while a vacuum is applied through the tube to the skin surface to remove cells abraded from the skin surface. The vacuum also causes the skin to be held in intimate contact with the abrasive tip during the treatment procedure.

The disposable tip system is the most crucial sub system. It can be easily replace with a different one, one that is designed for the treatment of different areas of skin. It dramatically lowers the probability of cross-infection among patients, without a significant cost.

1. An advanced method of treating the skin surface of a patient to remove surface cells and reduce undesirable skin blemishes comprising.

2. A skin treatment device/system for removing portions of the outer and undesired layers of skin.

3. The microdermabrasion system comprises a vacuum/supply housing, a hollow tube and an abrasive tip attached to a skin-contacting end.

4. The abrasive coated tip is moved over the skin surface while a vacuum, which causes the skin to be held in intimate contact with the abrasive tip during the treatment procedure, is applied through the tube to the skin surface to remove cells abraded from the skin surface.

5. The system includes three sub systems:
5.1 The vacuum/supply system is the system collects the scraps of the undesired outer skin layer and provide immediate oxygen supply to the new and flesh skin layer.
5.1.1 The vacuum system has a vacuum pump enclosed within a housing.
5.1.2 The vacuum housing has electronic means for monitoring and controlling the level of vacuum delivered. The air pressure of the nutrition can be adjusted through the vacuum/supply house.
5.1.3 The supply sub system provides nutrition such as oxygen to new skins. And it also helps the patient to take the procedure in a high oxygen environment and thus help the patient to relax during the procedure.
5.2 The disposable tip system comprises filters and a disposable abrasive tip.
5.2.1 The tip is removable and disposable.
5.2.2 The tip is mounted with natural or men-made abrasive material, created physically and chemically. (Because it is disposable, it minimizes the probability of cross-infection among patients.)
5.2.3 The tip system further includes manual or mechanical valve means for varying the level of reduced pressure applied through the treatment tip. This is a valve mechanism in operative connection to the source of vacuum.
5.2.4 On the surface of the working area of the abrasive tip device, the tip is mounted with men-made particles of diamond, aluminum oxide, silicon carbide, silicon oxide, metal nitrides or natural powders such as coconut shell powder. The working area of the tip can also be chemically created to have a roughened surface.
5.2.5 The tip system includes filters, which can be made of paper, cotton, metal, sponge, plastic and wood.
5.2.6 The tip has different shapes and models in order to work on different parts of the skins, to achieve the best polishing results.
5.2.7 The roughness of the surface of the tip has been classified into several levels. (For example, Level 1 tip is used to treat tender skin area, such as the area under the eyelids. Level 5 tip is to treat skin areas such as skin of cheeks. Level 10 is to treat the skin of body, etc.)
5.2.8 The disposable tip can have various shapes.
5.3 The tube system, which applies a negative pressure to a skin surface to be treated, via vacuum causing increased contact between the skin surface and the abrasive surface, comprises a tubular device with lumen for performing micro-abrasion of a skin surface.
5.3.1 The tubular device has a first end with a connection to the tip system, and a second end thereof for attachment to the vacuum system.
5.3.2 Inside the tube there are two channels: One to provide nutrition such as oxygen with positive air pressure; the other channel, which comprise a filter, provide a negative air pressure to collect the skin scraps. (Thus this system provides immediate nutrition supply such as oxygen or essences to the new and flesh skin layer when the vacuum system is collecting the scraps of the outer skin layer.)
5.3.3 The tube system includes optional manual or mechanical valve means for varying the level of reduced pressure applied through the treatment tube.
5.3.4 The device of the tube system includes a disposable collection filter positioned between the treatment tip and the source of vacuum so that all particulate matter entering the at least one opening in the abrasive treatment tip is collected therein.
